(19) United States
(12) Patent Application Publication
(10) Pub. No.: US 2004/0106758 A1
(43) Pub. Date: Jun. 3, 2004

(54) Curable polyvinylbenzyl compound and process for producing the same

(76) Inventors: Shouji Nishiguchi, Gunma (JP); Tatsuhiro Ikeya, Gunma (JP); Haruo Yoshida, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/474,453

(22) PCT Filed: Mar. 25, 2002
(86) PCT No.: PCT/JP02/02851

(30) Foreign Application Priority Data
Apr. 9, 2001 (JP) 2001-110125

(51) Int. Cl. C08F 120/06; C07C 321/24; C07C 43/21

(52) U.S. Cl. 526/317.1; 526/346; 568/58; 568/659; 570/184

(57) ABSTRACT

A curable polyvinyl benzyl compound represented by the following general formula (1):

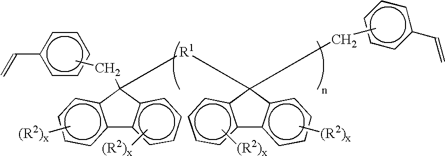

wherein R¹ represents a C₂₋₂₀ organic group, R² represents a hydrogen atom, etc., x is an integer of 0 to 4, and n is an integer of 0 to 2. The compound is obtained by reacting a fluorene compound with a vinylbenzyl halide in the presence of an alkali.

### TECHNICAL FIELD

The present invention relates to a compound which provides a cured product having high heat resistance, low water absorption and excellent dielectric properties which are required for organic insulating materials for use in electronic equipment such as communication equipment and to a process for producing the same. More specifically, the present invention relates to a curable polyvinyl benzyl compound obtained by reacting a fluorene compound with a vinylbenzyl halide, a process for producing the same, and a curable resin composition and a cured resin obtained by using the same. Further, the present invention relates to a substrate, a prepreg and a metal foil having a resin, all of which have excellent dielectric properties at a high-frequency range, in particular, a low dielectric dissipation factor, and high heat resistance.

### BACKGROUND ART

Along with recent progress in electronic technology, materials having a low dielectric constant and a low dielectric dissipation factor are now in demand as materials of parts for use in computers and mobile communication equipment. To satisfy this demand, various materials are being developed. The materials include, for example, polybenzocyclobutene (R. A. Kirchhoff et al., Macromol. Symp. 54/55, 531 (1992)), fluorinated polybiphenylene ether (JP 10-74751 A), polyphenylene compound having a heterocyclic side chain (JP 9-278879 A), polyfumarate (JP 9-208697 A), polynorbornene (JP 5-214079 A), polyquinoxaline (JP 2705799 B), fluorinated polyquinoline (JP 6-500591 A), side chain allyl group-substituted polyphenylene ether (JP 64-69628 A, JP 4-183707 A, and JP 6-207096 A), and polyphenylene ether whose terminal is blocked with an allyl group or a propargyl group (JP 7-51625 B).

However, the above materials proposed in the prior art have various problems such as a low crosslinking density and a large linear expansion coefficient; low chemical resistance; poor tenacity; a large number of complicated steps required for the production of a resin from raw materials; and the need for a special solvent for shaping. Therefore, they have not been put to practical use yet.

The inventors of the present invention have proposed a vinylbenzyl ether compound which has low water absorption over a wide temperature range and a wide frequency range, a low dielectric constant and a low dielectric dissipation factor and satisfies the current strict requirements for electronic materials (JP 9-31006 A). This vinylbenzyl ether compound can be synthesized by reacting an aromatic compound having a hydroxyl group with a vinylbenzyl halide in a polar solvent in the presence of an alkali, or in a water/organic solvent mixed solution in the presence of a phase-transfer catalyst.

However, the requirements for dielectric properties of electronic materials are becoming more and more demanding. Next-generation communication devices have begun to appear, which require, in particular, a low dielectric dissipation factor, which cannot be satisfied even by the above vinylbenzyl ether compound.

It is therefore an object of the present invention to provide a polyvinyl benzyl compound which provides a cured product having high heat resistance, low water absorption, a low dielectric constant and a low dielectric dissipation factor, and a process for producing the same.

It is another object of the present invention to provide a substrate, prepreg and metal foil having a resin all of which have excellent dielectric properties over a high frequency range, in particular, a low dielectric dissipation factor, and high heat resistance.

### DISCLOSURE OF THE INVENTION

The invention according to claim 1 relates to a curable polyvinyl benzyl compound represented by the following general formula 1:

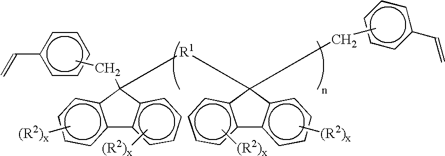

(wherein R¹ is a divalent organic group having 2 to 20 carbon atoms, R² is at least one organic group selected from the group consisting of a hydrogen atom, a halogen atom, an alkyl group, an alkoxy group and a thioalkoxy group having 1 to 5 carbon atoms, which may be the same or different, and an aryl group, where x is an integer of 0 to 4, and n is an integer of 0 to 20).

The invention according to claim 2 relates to a process for producing a curable polyvinyl benzyl compound according to claim 1, characterized by reacting one fluorene compound or two or more fluorene compounds represented by the following general formula 2 and a vinylbenzyl halide in the presence of an alkali:

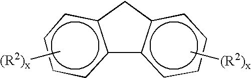

(wherein R² is at least one organic group selected from a group consisting of a hydrogen atom, a halogen atom, an alkyl group, an alkoxy group and a thioalkoxy group having 1 to 5 carbon atoms, which may be the same or different, and an aryl group, where x is an integer of 0 to 4).

The invention according to claim 3 relates to a process for producing a curable polyvinyl benzyl compound according to claim 1, characterized by reacting one fluorene compound or two or more fluorene compounds represented by the following general formula 2, a vinylbenzyl halide and a dihalomethyl compound having 2 to 20 carbon atoms in the presence of an alkali:

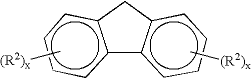

(wherein R² is at least one organic group selected from the group consisting of a hydrogen atom, a halogen atom, an alkyl group, an alkoxy group and a thioalkoxy group having 1 to 5 carbon atoms, which may be the same or different, and an aryl group, where x is an integer of 0 to 4).

The invention according to claim 4 relates to the process for producing a curable polyvinyl benzyl compound according to claim 2 or 3, in which vinylbenzyl halide is at least one selected from the group consisting of m-vinylbenzyl chloride and p-vinylbenzyl chloride.

The invention according to claim 5 relates to the process for producing a curable polyvinyl benzyl compound according to claim 3, in which an equivalent ratio of a halomethyl group of the vinylbenzyl halide to the halomethyl group of the dihalomethyl compound having 2 to 20 carbon atoms is 1.0/0 to 0.1/0.9.

The invention according to claim 6 relates to the process for producing a curable polyvinyl benzyl compound according to any one of claims 2 to 5, in which the reaction is carried out in the presence of an aprotic polar solvent and/or a phase-transfer catalyst.

The invention according to claim 7 relates to a curable resin composition prepared by mixing a curable polyvinyl benzyl compound according to claim 1 with a monomer, an oligomer and/or a polymer which is copolymerizable with said compound.

The invention according to claim 8 relates to a cured resin obtained by curing a curable polyvinyl benzyl compound according to claim 1.

The invention according to claim 9 relates to a cured resin obtained by curing a curable resin composition according to claim 7.

The invention according to claim 10 relates to a high-frequency substrate obtained by curing a curable polyvinyl benzyl compound according to claim 1.

The invention according to claim 11 relates to a high-frequency substrate obtained by curing a curable resin composition according to claim 7.

The invention according to claim 12 relates to a prepreg obtained by impregnating a curable resin composition according to claim 7 with a fiber material.

The invention according to claim 13 relates to a high-frequency substrate obtained by heating and pressurizing either a single prepreg according to claim 12 or a laminate of the prepregs according to claim 12.

The invention according to claim 14 relates to a metal-lined high-frequency substrate obtained by placing a metal foil onto either or single prepreg according to claim 12 or a laminate of the prepregs according to claim 12, through heating and pressurizing.

The invention according to claim 15 relates to a metal foil having a resin obtained by applying a curable resin composition according to claim 7 to a metal foil to be integrated.

The invention according to claim 16 relates to a multi-layer laminate substrate characterized by including a curable resin composition according to claim 7 applied to a conductive layer, which is polymerized and cured, and a conductive layer formed on a cured product.

### DETAILED DESCRIPTION OF THE INVENTION

The present invention will be described in detail hereinafter.

A curable vinylbenzyl compound of the present invention is obtained by reacting one fluorene compound or two or more fluorene compounds represented by the above general formula 2 with a vinylbenzyl halide and optionally a dihalomethyl compound having 2 to 20 carbon atoms in the presence of an alkali. The reaction can be carried out in accordance with conditions for a known vinylbenzylation reaction. The vinylbenzylation reaction is described, for example, by L. J. Mathias et al. in J. Polym. Sci., Part B; 36, 2869 (1998) and J. Polym. Sci., Part A; 35, 587 (1997), and by C. J. Kelly et al. in J. Chem. Res. (S), 446 (1997).

Examples of the fluorene compound used in the present invention include fluorene compounds whose fluorene and aromatic ring parts may be substituted by an alkyl group, alkoxy group, thioalkoxy group or aryl group as represented by the above general formula 2. They may be used alone or in combination of two or more compounds.

Examples of the vinylbenzyl halide used in the present invention include m-vinylbenzyl chloride, p-vinylbenzyl chloride, m-vinylbenzyl bromide and p-vinylbenzyl bromide. They may be used alone or in combination of two or more compounds. Of these, m-vinylbenzyl chloride and p-vinylbenzyl chloride are preferred.

The dihalomethyl compound used in the present invention is a compound having two —CH₂X (where X is a halogen atom) groups in the molecule and 2 to 20 carbon atoms, preferably 2 to 16 carbon atoms. Examples of the dihalomethyl compound include halogenated alkyls such as 1,2-dichloroethane, 1,2-dibromoethane, 1,3-dichloropropane, 1,3-dibromopropane, 1,4-dichlorobutane and 1,4-dibromobutane, and compounds such as o-xylylene dichloride, m-xylylene dibromide, p-xylylene dibromide, 4,4′-bis(chloromethyl)biphenyl, 4,4′-bis(chloromethyl)diphenyl ether, 4,4′-bis(chloromethyl)diphenyl sulfide, 2,6-bis(bromomethyl)naphthalene, 1,8-bis(bromomethyl)naphthalene and 1,4-bis(chloromethyl)naphthalene. They may be used alone or in combination of two or more compounds as far as an intramolecular cyclization reaction does not occur.

The equivalent ratio of the halomethyl group of the vinylbenzyl halide to the halomethyl group of the dihalomethyl compound can be selected as far as gelation is not caused by the dihalomethyl compound. The equivalent ratio of the vinylbenzyl halide to the dihalomethyl compound is preferably in the range of 1.0/0 to 0.1/0.9. When the amount of the vinylbenzyl halide is below the above range, curability deteriorates and the physical properties such as heat resistance of the cured product deteriorate.

Examples of the reaction solvent include aprotic polar solvents such as dimethylformamide, dimethyl sulfoxide, dimethyl acetamide, N-methylpyrrolidone, dioxane, acetonitrile, tetrahydrofuran, ethylene glycol dimethyl ether, 1,3-dimethoxypropane, 1,2-dimethoxypropane, tetramethylene sulfone, hexamethyl phosphamide, methyl ethyl ketone, methyl isobutyl ketone, acetone and cyclohexanone, and mixtures thereof. A solvent may be selected from among these according to the types of raw materials and reaction conditions so that a reaction system becomes uniform.

Examples of the alkali used in the present invention include alkoxides, hydrides and hydroxides of an alkali metal or alkali earth metal such as sodium methoxide, sodium ethoxide, sodium hydride, sodium borohydride, potassium hydride and potassium hydroxide. The alkali may be selected according to whether the reaction system is made hydrous or anhydrous.

The amount of the alkali is preferably 1.1 to 3.0 equivalents based on 1 equivalent of the hydrogen atom at the 9-position of the fluorene compound as a raw material. When the amount is less than 1.1 equivalents, the reaction rate becomes very low and the reaction does not proceed completely, with the result that the raw materials remain and an undesirable influence is exerted on the physical properties of the cured product. When the amount is beyond 3 equivalents in use, a large amount of a solvent for removing the residual alkali, such as washing water, must be used, which is not economical.

A phase-transfer catalyst may be used for the reaction in the present invention. Examples of this phase-transfer catalyst include onium salts such as quaternary ammonium compounds including tetra-n-butylammonium bromide, tetra-n-butylammonium hydrogen sulfate, benzyltrimethylammonium chloride and tricaprylmethylammonium chloride, quaternary phosphonium compounds including tetra-n-butylphosphonium bromide, benzyltriphenylphosphonium chloride, tetraphenylphosphonium chloride and tetraphenylphosphonium bromide, and tertiary sulfonium compounds such as benzyltetramethylene sulfonium bromide, and mixtures thereof.

The amount of the phase-transfer catalyst used cannot be completely specified because catalytic effect differs according to the type of catalyst or the reaction temperature. However, it is generally about 0.01 to 0.2 equivalent based on 1 equivalent of the hydrogen atom at the 9-position of the fluorene compound as a raw material.

The reaction temperature and reaction time cannot be completely specified because they differ according to the types of raw material compound and the reaction conditions but may be preferably 30 to 100° C. and 0.5 to 20 hours, respectively. When the reaction temperature is higher than 100° C., an unpreferred reaction such as thermal polymerization occurs and when the reaction temperature is lower than 30° C., though the reaction proceeds, it takes a long time, which is not economical.

Since a highly polymerizable unsaturated halide such as vinylbenzyl halide is used in the present invention, a thermal polymerization inhibitor may be optionally added to the reaction system. Examples thereof include t-butylcatechol, 2,4-di-t-butylphenol, 2-t-butylphenol, 2-t-butyl-4-nitrophenol, 2,4-dinitrophenol, hydroquinone, methyl hydroquinone, hydroquinone monomethyl ether, t-butylhydroquinone, resorcin, pyrogallol, phenothiazine or copper salt. Further, use of a suitable amount of air is effective in inhibiting polymerization.

The amount of the thermal polymerization inhibitor used cannot be completely specified because its effect differs according to the type of thermal polymerization inhibitor. However, an inhibitor of several ppm to 2,000 ppm based on the curable vinylbenzyl compound is sufficient.

The curable polyvinyl benzyl compound represented by the above general formula 1 of the present invention is obtained by the above production process. In the general formula 1, the divalent organic group of R¹ is derived from the carbon chain of the dihalomethyl compound. Also, n may be duly determined according to the desired degree of polymerization and mechanical strength and R² is determined according to the type of fluorene compound.

The curable polyvinyl benzyl compound of the present invention may be mixed with a monomer, oligomer and/or polymer copolymerizable with the above compound without departing from the gist of the present invention to prepare a curable resin composition having improved moldability. Specific examples of the monomer, oligomer and polymer include oligomers and polymers having a polymerizable unsaturated group, such as vinyl ester resins, unsaturated polyester resins, diallyl phthalate resins, maleimide resins and polycyanate resins of polyphenol, monomers and prepolymers such as triallyl isocyanurate and triallyl cyanurate, styrene, vinyltoluene, divinylbenzene, vinylbenzyl ether compounds, and monofunctional and polyfunctional (meth)acrylic acid derivative compounds.

The total use amount of the above copolymerizable monomer, oligomer and/or polymer cannot be completely specified because it differs according to the types thereof, compatibility with the vinylbenzyl compound and the intended application of the cured product. It is 0 to 300 parts by weight, preferably 0 to 200 parts by weight based on 100 parts by weight of the curable polyvinyl benzyl compound. It is more preferably 10 to 100 parts by weight. An addition amount beyond 300 parts by weight is undesirable because separation and exudation from the curable polyvinyl benzyl compound readily occur.

The curable polyvinyl benzyl compound and curable resin composition of the present invention can be cured by a known method such as heat, light or an electron beam. It is also useful to reduce the curing temperature or promote a curing reaction by using a curing agent. The cured product can be suitably used in organic insulating materials, etc. for use in electronic equipment such as communications equipment.

When a curing agent is used, benzoyl peroxide, cumene hydroperoxide, 2,5-dimethyl-2,5-di(t-butylperoxy)hexine-3,t-butylcumyl peroxide, methyl ethyl ketone peroxide, dicumyl peroxide or t-butyl perbenzoate for example, may be used according to the intended application.

The amount of the curing agent used differs according to the type and content of an unsaturated group contained in the curable polyvinyl benzyl compound or the curable resin composition, the type of the used curing agent, the half-life temperature and required stability, but is generally 0 to 10 parts by weight based on 100 parts by weight of the curable polyvinyl benzyl compound or the curable resin composition.

In addition, a known curing accelerator such as manganese naphthenate, lead naphthenate, zinc naphthenate, cobalt naphthenate, zinc octylate, dimethyl aniline or phenyl morpholine may also be used.

The curing temperature cannot be completely specified because it differs according to the type of the polymerizable unsaturated group and the type and amount of the curing agent used but it is 20 to 250° C., preferably 50 to 250° C. A curing temperature less than 20° C. is undesirable because curing may be insufficient.

A known curing retardant such as hydroquinone, benzoquinone or copper salt may be mixed to adjust curing conditions.

In addition, the curable polyvinyl benzyl compound and/or curable resin composition of the present invention may be optionally mixed with a colorant, filler and reinforcing fiber by a kneader, blender or roll to prepare a molding material or composite material. Silica, alumina, zirconia, titanium dioxide, magnesium hydroxide, aluminum hydroxide or calcium carbonate may be added as the filler without departing from the gist of the present invention.

The above curable polyvinyl benzyl compound or curable resin composition is molded in a desired shape to obtain a high-frequency substrate of the present invention. The high-frequency substrate of the present invention is suitable for use at a high frequency range of 100 MHz or higher, in particular, 1 GHz or higher. The dielectric dissipation factor can be maintained at about 0.002 to 0.01 at this high frequency range.

The present invention further provides a prepreg which is obtained by impregnating the above curable resin composition with a fiber material.

A known fiber material such as glass fiber, carbon fiber, aromatic polyamide fiber, silicon carbide fiber or alumina fiber may be used as the fiber material used in the preparation of the prepreg of the present invention. It is preferably glass cloth formed from a glass fiber having low dielectric properties (a low dielectric constant and a low dielectric dissipation factor). A fiber material content of 30 to 70 wt % based on the prepreg is preferable from the viewpoints of strength and moldability.

In the present invention, to impregnate the curable resin composition with the fiber material, either a known solvent method or a solvent-free method may be used. As for the solvent to be used in the solvent method, a solvent having a relatively low boiling point such as a ketone-based solvent exemplified by acetone, methyl ethyl ketone and methyl isobutyl ketone, or an aromatic hydrocarbon-based solvent exemplified by benzene and toluene may be used in order to reduce the amount of the residual solvent contained in the prepreg as much as possible and avoid a reduction in heat resistance, cracking or the formation of voids.

A prepreg can be obtained by drying and heating the fiber material into which the curable resin composition is impregnated by the above method at 80 to 130° C. for 10 to 180 minutes as required.

A high-frequency substrate can be obtained by heating and pressurizing the obtained single prepreg or a laminate of the prepregs. That is, the high-frequency substrate can be obtained by molding a single prepreg having a predetermined thickness or a laminate of prepregs having a predetermined total thickness by applying heat and pressure in accordance with a known method such as thermal pressing. The molding conditions include a temperature of 80 to 250° C., preferably 100 to 200° C., a pressure of 5 to 100 kg/cm², and a time of 0.5 to 10 hours, for example. It is also effective to increase the temperature stepwise as required.

The present invention also provides a metal-lined high-frequency substrate which is obtained by placing a metal foil on the above prepreg alone or the laminate of the prepregs and applying heat and pressure. That is, a metal-lined high-frequency substrate can be obtained by placing a metal foil on both sides of a single prepreg having a predetermined thickness or a laminate of prepregs having a predetermined total thickness and molding it by applying heat and pressure as described above.

The metal foil used in the present invention can be copper, gold, silver or aluminum foil but is preferably a copper foil. An electrolytic foil or rolled foil may be optionally used.

Further, by applying the above curable resin composition or a solution thereof to a metal foil such as the above copper foil using a doctor blade coating or the like, and drying and heating it at 80 to 130° C. for 10 to 180 minutes, it is also possible to obtain a metal foil having a resin wherein both the foil and the resin composition (or a solution thereof) are integrated. This metal foil may then be used as a high-frequency substrate. A multi-layer laminate substrate may be produced by placing this metal foil having a resin on a core material and molding it by applying heat and pressure.

According to the present invention, there is provided a multi-layer laminate substrate obtained by applying the above curable resin composition onto a conductive layer, polymerizing and curing the composition and further forming a conductive layer on the cured product.

This multi-layer laminate substrate can be manufactured by a so-called build-up process in which an 18 μm-thick copper foil is used as a conductive layer, the curable resin composition is applied onto the conductive layer with a thickness of 20 to 200 μm, preferably 50 to 100 μm as an insulating layer, and thermally cured, and further a conductive layer is formed on the cured product.

The prepreg and the metal foil having a resin obtained by using the above curable resin composition have been described above. The curable vinylbenzyl compound of the present invention may be used in place of the curable resin composition.

### BRIEF DESCRIPTION OF THE DRAWINGS

FIG. 1 shows an ¹H-NMR spectrum of a compound 1 obtained in Example 1;

FIG. 2 shows an IR spectrum of the compound 1 obtained in Example 1;

FIG. 3 shows the ¹H-NMR spectrum of a compound 2 obtained in Example 2;

FIG. 4 shows the IR spectrum of the compound 2 obtained in Example 2; and

FIG. 5 shows the ¹H-NMR spectrum of a compound 5 obtained in Example 4.

### BEST MODE FOR CARRYING OUT THE INVENTION

Hereinafter, the present invention will be described based on examples and comparative examples. However, the present invention is not limited to those examples. The term “parts” in the examples means “parts by weight” unless otherwise stated. Measurement methods carried out in Examples 1 to 4 and Comparative Examples 1 and 2 are shown below.

(1) Weight reduction start temperature: measured under a nitrogen flow at a temperature increase rate of 10° C./min using TG/DTA6200 available from SII Co., Ltd.

(2) Dielectric properties: measured using the 4285A and 4285B LCR meters available from Yokogawa Hewlett Packard Co., Ltd. in accordance with an equilibrium bridge method (1 MHz).

(3) ¹H-nuclear magnetic resonance spectrum (¹H-NMR): measured using tetramethylsilane as an internal standard material and the JNM-LA300 available from JEOL Ltd.

(4) IR spectrum: measured using the Fourier Transform Infrared Spectrophotometer, JIR-RFX3002 FT-IR SPECTROPHOTOMETER available from JEOL Ltd.

(5) Gel permeation chromatography (GPC): The molecular weight (Mw) in terms of standard polystyrene was measured at a column temperature of 40° C. and an elution rate of 1 ml/min using tetrahydrofuran as an eluate and the Shodex GPC System-21 (column KF-802, KF-803, KF-805) available from Showa Denko K.K.

(6) Water absorption: calculated from dry weight and weight after the absorption of water by immersing a test specimen measuring 1.5 mm×50 mm×50 mm in water at 25° C. for 24 hours.

### EXAMPLE 1

49.8 g (0.3 mol) of fluorene, 200 g of methylisobutyl ketone, 2.91 g (9×10⁻³ mol) of tetra-n-butylammonium bromide, 0.73 g of hydroquinone and 96 g of a 50 wt % aqueous solution of NaOH (NaOH purity of 95%, 1.14 mol) were charged into a 1-liter four-necked flask equipped with a thermoregulator, stirrer, cooling condenser and dropping funnel and heated at 62° C. under agitation to prepare a uniform solution. 117 g of vinylbenzyl chloride CMS-AM (m-/p-isomers: 50/50 wt % mixture) available from Seimi Chemical Co., Ltd. (purity of 91%, 0.7 mol) was added dropwise to this dark blue green solution over 20 minutes and then a reaction was carried out at 60 to 61° C. for 7 hours. After 200 ml of toluene was added to the obtained green reaction product, the obtained solution was neutralized with 2N hydrochloric acid and washed with distilled water three times, toluene was removed under reduced pressure, and the obtained light yellow viscous solid was recrystallized from fresh toluene to obtain 73.4 g of a gray white solid having a melting point measured by DSC of 142° C. (yield of 61.5%). This is designated as compound 1.

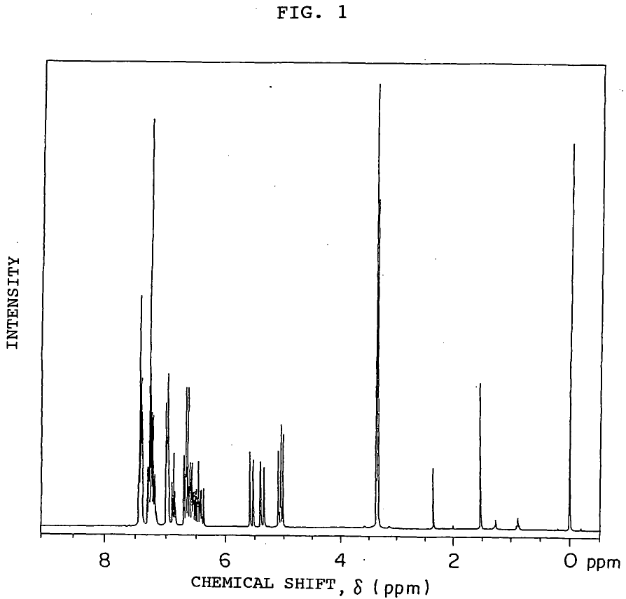

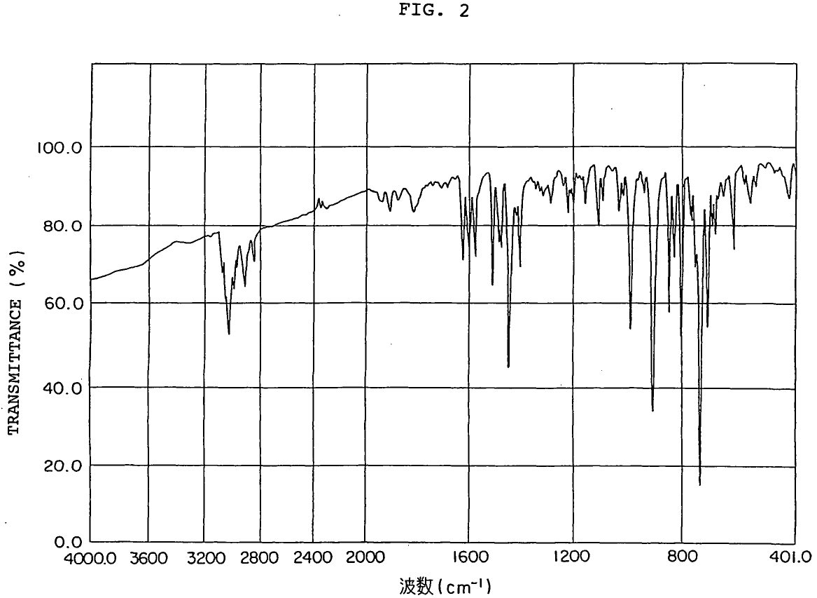

Compound 1 was identified from its ¹H-NMR spectrum, IR spectrum and GPC measurement. FIG. 1 shows the ¹H-NMR spectrum and FIG. 2 shows the IR spectrum. It was found from the GPC measurement results that the product had an Mw of 400 and it was judged from these measurement results that the product was 9,9-bis(vinylbenzyl)fluorene (in the general formula 1, R² is a hydrogen atom and n=0).

The compound 1 was placed in a mold heated at 150° C. and press-cured at 150° C. with a pressure of 4.9 MPa to 7.8 MPa (50 to 80 kgf/cm²) for 1 hour and at 180° C. with the same pressure for 5 hours to manufacture a resin plate so as to prepare test specimens required for each measurement. The measurement results are shown in Table 1.

### EXAMPLE 2

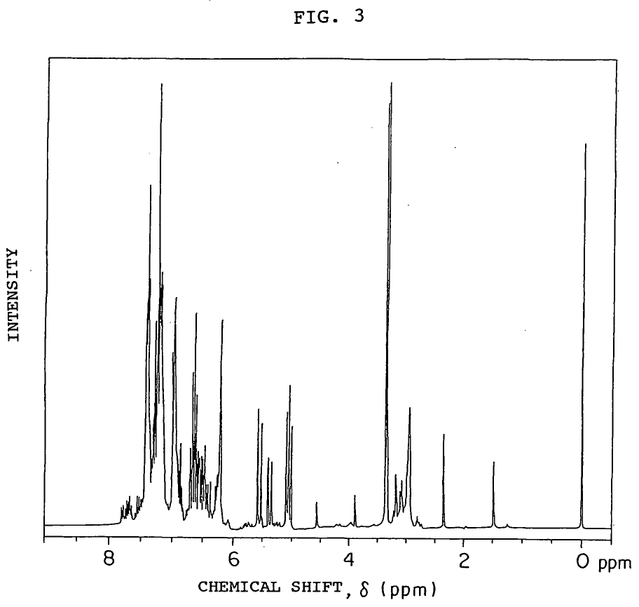

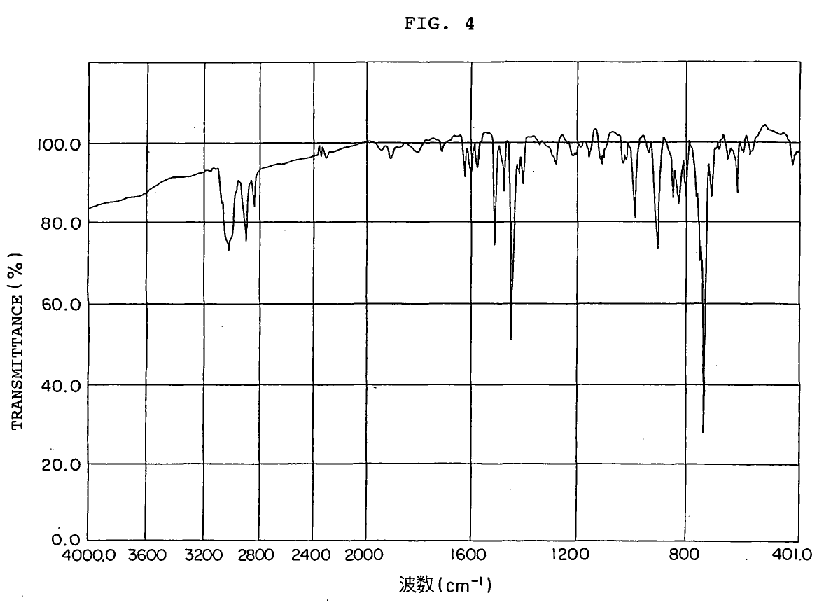

49.8 g (0.3 mol) of fluorene, 220 g of toluene, 2.91 g (9×10⁻³ mol) of tetra-n-butylammonium bromide and 96 g of a 50 wt % aqueous solution of NaOH (purity of 95%, 1.14 mol) were added to the reactor used in Example 1 and heated at 65° C., and 21 g (0.12 mol) of p-xylylene dichloride was added, and reacted for 2.5 hours. After it was confirmed from the results of the ¹H-NMR measurement of a small amount of the reaction product that p-xylylene dichloride was consumed, 54 g of CMS-AM (purity of 91%, 0.36 mol) was added dropwise to the reaction system and the reaction was continued at 65° C. for 6.5 hours. After the reaction solution was cooled to room temperature, 2N hydrochloric acid was added to neutralize the reaction mixture, and distilled water was added to the organic layer, which was then washed three times. After the solvent was distilled off under reduced pressure, the obtained solid was pulverized and filtered in methanol to collect solid matter through filtration, which was then dried at 50° C. in a vacuum oven to obtain a curable polyvinyl benzyl compound at a yield of 90%. The molecular weight Mw measured by GPC of the compound was 3,100. The melting point measured by DSC of the compound was 75 to 120° C. This is designated as compound 2. FIG. 3 shows the ¹H-NMR spectrum of this compound and FIG. 4 shows the IR spectrum of the compound. Compound 2 is a compound of the general formula 1 in which R¹ is a xylylene group, R² is a hydrogen atom and n=about 10 (mixed with a compound in which n=0).

Compound 2 was then poured into the gap between glass plates and cured at 130° C. for 2 hours, at 160° C. for 2 hours and after-cured at 180° C. for 5 hours. Test specimens required for each measurement were prepared from the obtained resin plate. The measurement results are shown in Table 1.

### EXAMPLE 3

A solution containing 60 wt % of Compound 2 synthesized in Example 2 and 40 wt % of divinyl benzene (purity of 82%) was prepared, poured into the gap between glass plates and cured at 100° C. for 6 hours, at 160° C. for 4 hours and after-cured at 180° C. for 2 hours. Test specimens required for each measurement were prepared from the obtained resin plate. The results are shown in Table 1.

### COMPARATIVE EXAMPLE 1

45 g (0.25 equivalent) of dicyclopentadiene skeleton phenolic resin, DPP-3H (special phenolic resin manufactured by Nippon Petrochemical Co., Ltd.), 38.1 g of vinylbenzyl chloride CMS-AM (m-/p-isomers: 50/50 wt % mixture) (purity of 91%, 0.25 mol), 2.4 g of tetra-n-butylammonium bromide, 0.038 g of 2,4-dinitrophenol and 200 g of methyl ethyl ketone were charged into a 1-liter four-necked flask equipped with a thermoregulator, stirrer, cooling condenser and dropping funnel and dissolved under agitation, and 40 g of a 50 wt % aqueous solution of NaOH (NaOH purity of 95%, 0.475 mol) was added dropwise at 75° C. to the obtained solution over 20 minutes and further stirred at 75° C. for 4 hours. After the obtained reaction mixture was cooled to room temperature, it was neutralized with 2N hydrochloric acid, 100 g of toluene was added, and the organic layer was then washed three times with 300 g of distilled water. After methyl ethyl ketone was removed under reduced pressure, the reaction product was precipitated in 300 ml of methanol to collect solid matter through filtration, which was then dried at 50° C. in a vacuum oven to obtain a vinylbenzyl ether compound at a yield of 95%. This is designated as compound 3.

Compound 3 was cured and molded in the same manner as in Example 1 to prepare a resin plate. Test specimens required for each measurement were prepared from this resin plate. The measurement results are shown in Table 1.

### COMPARATIVE EXAMPLE 2

2 parts of 2-ethyl-4-methylimidazole (available from Shikoku Kasei Co., Ltd.) was mixed with 100 parts of an epoxy resin (Epicoat 828, available from Yuka Shell Epoxy Co., Ltd. (epoxy equivalent 188), to prepare a resin composition. This is designated as compound 4.

Compound 4 was poured into the gap between glass plates and cured at 80° C. for 2 hours and after-cured at 150° C. for 2 hours to manufacture a resin plate. Test specimens required for each measurement were prepared from this resin plate. The measurement results are shown in Table 1.

### EXAMPLE 4

54.1 g (0.3 mol) of 1-methylfluorene, 200 g of methylisobutyl ketone, 2.91 g (9×10⁻³ mol) of tetra-n-butylammonium bromide, 0.73 g of hydroquinone and 96 g of a 50 wt % aqueous solution of NaOH (NaOH purity of 95%, 1.14 mol) were charged into a 1-liter four-necked flask equipped with a thermoregulator, stirrer, cooling condenser and dropping funnel and heated at 62° C. under agitation to prepare a uniform solution. 117 g of vinylbenzyl chloride, CMS-AM (m-/p-isomers: 50/50 wt % mixture) available from Seimi Chemical Co., Ltd. (purity of 91%, 0.7 mol) was added dropwise to this dark blue green solution over 20 minutes, which was then reacted at 60 to 61° C. for 7 hours. After 200 ml of toluene was added to the obtained green reaction product, the obtained solution was neutralized with 2N hydrochloric acid and washed three times with distilled water, toluene was removed under reduced pressure, and the obtained light yellow viscous solid was recrystallized from fresh toluene to obtain 75.1 g of a gray white solid having a melting point measured by DSC of 142° C. (yield of 60.8%). This is designated as compound 5.

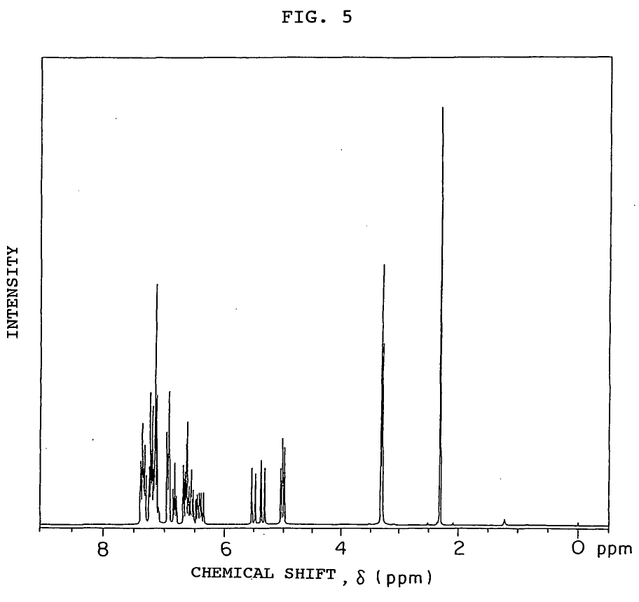

Compound 5 was identified from its ¹H-NMR spectrum, IR spectrum and GPC measurement. FIG. 5 shows the ¹H-NMR spectrum. It was found from the GPC measurement results that the product had an Mw of 410 and it was judged from these measurement results that the product was 1-methyl-9,9-bis(vinylbenzyl)fluorene.

Compound 5 was placed in a mold heated at 150° C. and press-cured at 150° C. with a pressure of 50 to 80 kgf/cm² for 1 hour and at 180° C. with the same pressure for 5 hours to manufacture a resin plate so as to prepare test specimens required for each measurement. The measurement results are shown in Table 1.

TABLE 1

| | | | | Comparative | Comparative | |
|---|---|---|---|---|---|---|
| | Example | Example | Example | Example | Example | Example |
| Mixing ratio | 1 | 2 | 3 | 1 | 2 | 4 |
| Compound 1 | 100 | | | | | |
| | parts | | | | | |
| Compound 2 | | 100 | 60 | | | |
| | | parts | parts | | | |
| Compound 3 | | | | 100 parts | | |
| Compound 4 | | | | | 100 parts | |
| Compound 5 | | | | | | 100 |
| | | | | | | parts |
| Divinylbenzene | | | 40 parts | | | |
| Cured product | | | | | | |
| physical | | | | | | |
| properties | | | | | | |
| 5% weight | 392° C. | 364° C. | 378° C. | 371° C. | 399° C. | 386° C. |
| reduction | | | | | | |
| temperature | | | | | | |
| Water | 0.12% | 0.11% | 0.12% | 0.16% | 1.4% | 0.14% |
| absorption | | | | | | |
| Dielectric | 2.65 | 2.67 | 2.79 | 2.82 | 3.29 | 2.69 |
| constant | | | | | | |
| (1 MHz) | | | | | | |
| Dielectric | 0.0013 | 0.0023 | 0.0017 | 0.0070 | 0.0249 | 0.0015 |
| dissipation | | | | | | |
| factor (1 MHz) | | | | | | |

It is understood from the results of Table 1 that the curable polyvinyl benzyl compound of the present invention attains better dielectric properties (lower dielectric constant and lower dielectric dissipation factor) than the conventional resins of the comparative examples without impairing heat resistance and has stable dielectric properties because of its lower water absorption.

### EXAMPLE 5

The glass cloth, WEA18K105BZ2 (available from Nitto Boseki Co., Ltd.) was impregnated with a 60% toluene solution of Compound 1 and dried at 120° C. for 60 minutes to obtain a prepreg. A 10-ply laminate of the prepregs was prepared and molded through the application of heat and pressure (40 kg/cm²) at 150° C. for 2 hours, at 180° C. for 5 hours and at 200° C. for 5 hours to obtain a laminated plate having a thickness of 1.6 mm and a glass fiber content of 60%.

The dielectric properties and solder heat resistance of this laminated plate were tested by the methods desirable below. As a result, the plate had a dielectric constant of 4.0, a dielectric dissipation factor of 0.0035 and a solder heat resistance of 120 seconds or more.

Dielectric properties: The dielectric constant and dielectric dissipation factor at 5 GHz of a prismatic specimen measuring 1.6 mm×1.5 mm×75 mm were measured by the vector network analyzer, HP8753E available from Hewlett Packard Co., Ltd. in accordance with a cavity resonator perturbation method.

Solder heat resistance test: In accordance with JIS C 0054,the specimen was immersed in a solder bath at 260° C. for 120 seconds to check if there was any change in its surface state or shape.

### EXAMPLE 6

The procedure of Example 5 was repeated except that the compound 2 was used in place of compound 1. As a result, the compound had a dielectric constant of 4.0, a dielectric dissipation factor of 0.0040 and a solder heat resistance of 120 seconds or more.

### EXAMPLE 7

The procedure of Example 5 was repeated except that the compound 5 was used in place of compound 1. As a result, the compound had a dielectric constant of 4.0, a dielectric dissipation factor of 0.0038 and a solder heat resistance of 120 seconds or more.

### EXAMPLE 8

A resin solution prepared by dissolving 100 parts of compound 1 and 120 parts of compound 2 in 80 parts of toluene was applied to a 35 μm-thick copper foil 3EC available from Mitsui Mining & Smelting Co., Ltd. to a thickness of 100 μm, dried at 100° C. for 60 minutes and heated at 120° C. for 2 hours to obtain a semi-cured product (two products were manufactured). These two copper foils having a resin were overlapped in such a manner that their resins were brought into contact with each other and molded through the application of heat and pressure at 150° C. for 2 hours and at 180° C. for 6 hours (40 kg/cm²) to obtain a specimen. In accordance with JIS C 6481, this specimen was then used to measure the copper foil's peel strength, which was 1.2 kgf/cm.

### INDUSTRIAL APPLICABILITY

According to the present invention, there is provided a polyvinyl benzyl compound which provides a cured product having high heat resistance, low water absorption, a low dielectric constant and a low dielectric dissipation factor, and a process for producing it.

Further, according to the present invention, there are provided a substrate, a prepreg, and a metal foil having a resin all of which have excellent dielectric properties at a high frequency range, in particular, a low dielectric dissipation factor, and high heat resistance.

1. A curable polyvinyl benzyl compound represented by the following general formula 1: 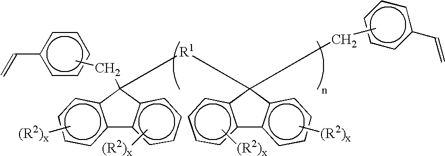
(wherein R¹ is a divalent organic group having 2 to 20 carbon atoms, R² is at least one organic group selected from the group consisting of a hydrogen atom, a halogen atom, an alkyl group, an alkoxy group and a thioalkoxy group having 1 to 5 carbon atoms, which may be the same or different, and an aryl group, where x is an integer of 0 to 4, and n is an integer of 0 to 20):

2. A process for producing a curable polyvinyl benzyl compound according to claim 1, characterized by reacting one fluorene compound or two or more fluorene compounds represented by the following general formula 2 and a vinylbenzyl halide in the presence of an alkali: 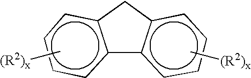
(wherein R² is at least one organic group selected from the group consisting of a hydrogen atom, a halogen atom, an alkyl group, an alkoxy group and a thioalkoxy group having 1 to 5 carbon atoms, which may be the same or different, and an aryl group, where x is an integer of 0 to 4).

3. A process for producing a curable polyvinyl benzyl compound according to claim 1, characterized by reacting one fluorene compound or two or more fluorene compounds represented by the following general formula 2, a vinylbenzyl halide and a dihalomethyl compound having 2 to 20 carbon atoms in the presence of an alkali: 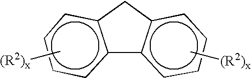
(wherein R² is at least one organic group selected from the group consisting of a hydrogen atom, a halogen atom, an alkyl group, an alkoxy group and a thioalkoxy group having 1 to 5 carbon atoms, which may be the same or different, and an aryl group, where x is an integer of 0 to 4).

4. A process for producing a curable polyvinyl benzyl compound according to claim 2 or 3, wherein the vinylbenzyl halide is at least one selected from the group consisting of m-vinylbenzyl chloride and p-vinylbenzyl chloride.

5. A process for producing a curable polyvinyl benzyl compound according to claim 3, wherein an equivalent ratio of a halomethyl group of the vinylbenzyl halide to the halomethyl group of the dihalomethyl compound having 2 to 20 carbon atoms is 1.0/0 to 0.1/0.9.

6. A process for producing a curable polyvinyl benzyl compound according to any one of claims 2 to 5, wherein the reaction is carried out in the presence of an aprotic polar solvent and/or a phase-transfer catalyst.

7. A curable resin composition prepared by mixing a curable polyvinyl benzyl compound according to claim 1 with a monomer, an oligomer and/or a polymer which is dopolymerizable with said compound.

8. A cured resin obtained by curing a curable polyvinyl benzyl compound according to claim 1.

9. A cured resin obtained by curing a curable resin composition according to claim 7.

10. A high-frequency substrate obtained by curing a curable polyvinyl benzyl compound according to claim 1.

11. A high-frequency substrate obtained by curing a curable resin composition according to claim 7.

12. A prepreg obtained by impregnating a curable resin composition according to claim 7 with a fiber material.

13. A high-frequency substrate obtained by heating and pressurizing either a single prepreg according to claim 12 or a laminate of the prepregs according to claim 12.

14. A metal-lined high-frequency substrate obtained by placing a metal foil onto either a single prepreg according to claim 12 or a laminate of the prepregs according to claim 12, through heating and pressurizing.

15. A metal foil having a resin obtained by applying a curable resin composition according to claim 7 to a metal foil to be integrated.

16. A multi-layer laminate substrate characterized by having a curable resin composition according to claim 7 applied to a conductive layer, which is polymerized and cured, and a conductive layer formed on a cured product.
